(19) United States
(12) Patent Application Publication
(10) Pub. No.: US 2005/0075711 A1
(43) Pub. Date: Apr. 7, 2005

(54) Balloon catheter with selectable diameter and expandable length

(76) Inventor: Anthony J. Neary, Galway (IE)

Correspondence Address: MEDTRONIC VASCULAR, INC.;IP LEGAL DEPARTMENT, 3576 UNOCAL PLACE, SANTA ROSA, CA, 95403, US

(21) Appl. No.: 10/678,833

(22) Filed: Oct. 3, 2003

(51) Int. Cl. A61F 2/06

(52) U.S. Cl. 623/1.11; 606/194

(57) ABSTRACT

One aspect of the invention provides a system for treating a vascular condition, including a catheter body, an inflatable balloon disposed on the catheter body, a plurality of concentrically disposed restrictors circumferentially disposed on the inflatable balloon, and inflation means coupled to a proximal end of the catheter body to inflate the inflatable balloon. One or more restrictors are at least partially removable to allow expansion of the inflatable balloon to a predetermined size. A method of treating a vascular condition and a balloon catheter including a plurality of restrictors is also disclosed.

### FIELD OF THE INVENTION

This invention relates generally to balloon catheters and stent-deployment systems. More specifically, the invention relates to restrictors for pre-selectively controlling the diameter and length of an inflatable balloon.

### BACKGROUND OF THE INVENTION

Medical balloon catheters are used in procedures to treat a wide variety of blood vessel disorders such as intravascular restrictions due to atherosclerosis or restenosis. Various techniques have been used to control, expand, or alter the size of catheter balloons that are used in angioplasty and stent placement procedures. The size of a catheter balloon and the amount of inflation fluid used to expand that balloon can give only limited control over the diameter and length of the deployed catheter balloon. Various balloon configurations as well as limiters such as sheaths have been used to provide greater control.

Some control techniques have required a catheter balloon to expand to a certain amount of predilation before fully inflating. For example, balloon expansion methods and systems have been developed for first expanding a stent to a relatively small diameter to open a lesion and then further expanding the stent to embed the stent in a vessel wall. Such methods are described in “Stent Installation Method Using Balloon Catheter Having Stepped Compliance Curve,” Wang, U.S. Pat. No. 6,402,778 issued Jun. 11, 2002 and “Stent Installation Method Using Balloon Catheter Having Stepped Compliance Curve,” Wang, U.S. Pat. No. 6,352,551 issued Mar. 5, 2002. The methods allow predilation of a balloon at a low pressure and predetermined diameter, followed by expansion of the balloon at a substantially larger diameter by high pressure.

A catheter balloon that is designed to be expanded to two different, known, work-hardened diameters is described in “Balloon Catheter and Inflation Method,” Miller, U.S. Pat. No. 5,779,730 issued Jul. 14, 1998. An associated method increasingly pressurizes a catheter balloon made of work-hardenable material for elastically expanding the balloon to such a first pressure that sufficient work-hardening takes place in the expanding balloon, causing the balloon diameter to substantially stop expanding even in response to a further increase in pressure.

Methods and systems have been developed to adjust the length of a catheter balloon. For example, two or more chambers of a balloon can be inflated independently, as disclosed in “Angioplasty Catheter System with Adjustable Balloon Length,” Lee et al., U.S. Pat. No. 6,527,741 issued Mar. 4, 2003 and “Adjustable Length Balloon Catheter,” Peacock, III et al., U.S. Pat. No. 5,549,551 issued Aug. 27, 1996.

Another approach to controlling the diameter and length of a catheter balloon is to introduce one tubular and elongate sheath or sleeve over a balloon or intravascular devices such as a stent. Sheaths have been introduced to provide greater control over the expansion of an expandable stent, as well as the inflation of a balloon catheter, as exemplified in “Peeling Sheath for Self-Expanding Stent,” Bigus et al., U.S. Patent Application 2003/0004561 published Jan. 2, 2003. A sheath of shape-memory polymer may be used help deploy and control expansion size, as described in “Expandable Introducer Sheath,” Kratoska et al., U.S. Pat. No. 6,183,443 issued Feb. 6, 2001. The sheath also may be manipulated while in a body vessel to expand its inner diameter to a larger diameter.

Another sheath-like device used to control the length of a balloon is described in “Captured Sleeve and Stent Delivery Device,” Amann et al., U.S. Pat. No. 6,066,155 issued May 23, 2000. A covering sleeve, which is movable along the catheter shaft, has a first portion with a first inner diameter surrounding the catheter shaft and a second portion with a second larger inner diameter being pushable over the deflated dilatation balloon. The covering sleeve provides some adjustability of the balloon length.

Mickley and others disclose another type of sleeve that adjusts the length of a catheter balloon in “Catheter Having a Variable Length Balloon and Method of Using the Same,” U.S. Pat. No. 5,961,536 issued Oct. 5, 1999. The outer sleeve includes a distal end that can be varied in size and configured to restrict inflation proximal the distal end. An earlier example of a tubular sheath used to alter or limit the expansion of an inflatable catheter balloon is disclosed in “Balloon Sheath”, Stone et al., U.S. Pat. No. 5,843,027 issued Dec. 1, 1998.

Guide tubes have been suggested for controlling the expansion of a catheter balloon, as taught in “Method and Catheter System for Delivering Medication with an Everting Balloon Catheter,” Johnson et al., U.S. Pat. No. 6,039,721 issued Mar. 21, 2000. The guide-tube system employs an annular catheter balloon having its proximal end secured to the lumen tube of a balloon catheter and its distal end secured to a guide tube. Relative axial movement of guide tubes, which define a lumen therebetween, adjusts the balloon between retracted and extended positions.

While the above-mentioned devices and systems provide some control over the diameter and length of a catheter balloon, an improved balloon catheter system would allow a physician the option of selecting whatever diameter or length of catheter balloon that may be needed either prior to or during a medical procedure. In addition, a preferred catheter balloon system would allow a balloon to be used once or multiple times during a procedure. A balloon catheter system having a variably sized balloon could be used for a wider range of vascular sizes and applications than those with a single size option. With the availability of a variably sized catheter balloon, the inventory of sized balloon catheters required in a medical treatment facility could be reduced.

### SUMMARY OF THE INVENTION

One aspect of the invention provides a system for treating a vascular condition, including a catheter body, an inflatable balloon disposed on the catheter body, a plurality of concentrically disposed restrictors circumferentially disposed on the inflatable balloon, and inflation means coupled to a proximal end of the catheter body to inflate the inflatable balloon. One or more restrictors are at least partially removable to allow expansion of the inflatable balloon to a predetermined size.

Another aspect of the invention provides a method of treating a vascular condition. A variable expansion balloon catheter including a plurality of concentrically disposed restrictors circumferentially disposed on an inflatable balloon is provided. At least one of the restrictors is selected based on a predetermined balloon size. The selected restrictors are axially displaced with respect to the inflatable balloon, and the inflatable balloon is inflated to the predetermined balloon size based on the axial displacement of the selected restrictors.

Another aspect of the invention is a balloon catheter including a catheter body, an inflatable balloon disposed on the catheter body, and a plurality of concentrically disposed restrictors circumferentially disposed on the inflatable balloon. One or more restrictors are axially translatable to allow expansion of the inflatable balloon to a predetermined size.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings.

### BRIEF DESCRIPTION OF THE DRAWINGS

Various embodiments of the present invention are illustrated by the accompanying figures, wherein:

FIG. 1 is an illustration of a system for treating a vascular condition, in accordance with one embodiment of the current invention;

FIG. 2a shows a longitudinal cross-sectional view of a portion of a balloon catheter with a plurality of concentrically disposed restrictors, in accordance with one embodiment of the current invention;

FIG. 2b, FIG. 2c, FIG. 2d, and FIG. 2e show longitudinal cross-sectional views of a portion of a balloon catheter with a varying number of concentrically disposed restrictors as an inflatable balloon is inflated, in accordance with one embodiment of the current invention;

FIG. 3a shows a diametrical cross-sectional view of a portion of a balloon catheter with a plurality of concentrically disposed restrictors, in accordance with one embodiment of the current invention;

FIG. 3b, FIG. 3c, FIG. 3d, and FIG. 3e show diametrical cross-sectional views of a portion of a balloon catheter with a plurality of concentrically disposed restrictors and an inflated balloon, in accordance with one embodiment of the current invention;

FIG. 4 shows a graph of balloon diameter with applied pressure for a variable expansion balloon catheter with a preselected outer diameter, in accordance with one embodiment of the current invention;

FIG. 5a, FIG. 5b, FIG. 5c, FIG. 5d, and FIG. 5e show longitudinal cross-sectional views of a portion of a balloon catheter with a plurality of axially translatable restrictors, in accordance with one embodiment of the current invention;

FIG. 6 shows a graph of balloon diameter versus position along the inflated balloon for a variable expansion balloon catheter with a preselected outer diameter and a preselected expandable length, in accordance with one embodiment of the current invention;

FIG. 7a, FIG. 7b, FIG. 7c and FIG. 7d show longitudinal cross-sectional views of a portion of a variable expansion balloon catheter with a stent for controlled-diameter stent delivery, in accordance with one embodiment of the current invention;

FIG. 8 shows a graphical illustration of stent diameter for a stent deployed with a variable expansion balloon catheter, in accordance with one embodiment of the current invention; and

FIG. 9 is a flow chart of a method for treating a vascular condition, in accordance with one embodiment of the current invention.

### DETAILED DESCRIPTION OF THE PRESENTLY PREFERRED EMBODIMENTS

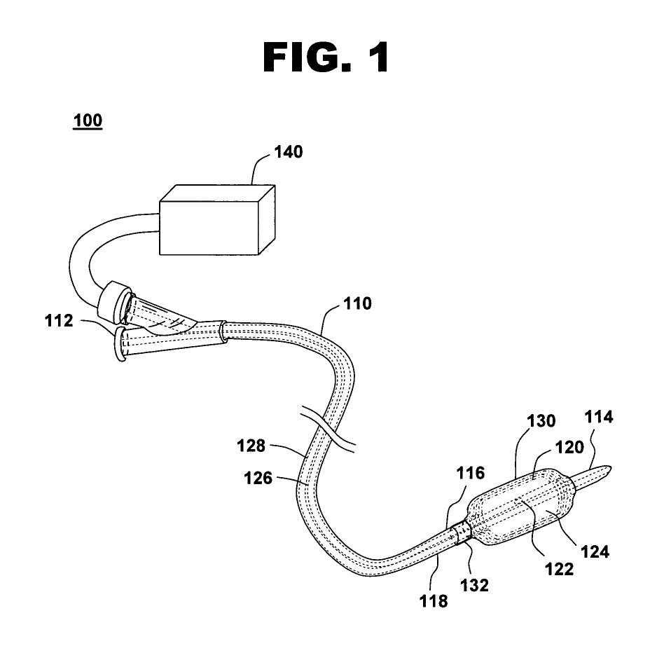

FIG. 1 illustrates a system for treating a vascular condition, in accordance with one embodiment of the present invention at 100. Vascular treatment system 100 includes a balloon catheter having a catheter body 110, an inflatable balloon 120 disposed on the catheter body, a plurality of concentrically disposed restrictors 130 circumferentially disposed on inflatable balloon 120, and inflation means 140 such as a balloon inflation system to inflate inflatable balloon 120. Restrictors 130 are axially translatable or at least partially removable to allow expansion of inflatable balloon 120 to a predetermined size. Restrictors 130 are axially translatable or at least partially removable to allow expansion to a predetermined balloon diameter or to control an expandable length of inflatable balloon 120. A practitioner may pre-select, for example, the balloon diameter and the expandable length by moving or removing one or more restrictors 130 from inflatable balloon 120 immediately prior to insertion into the body. The removal of all restrictors 130 from inflatable balloon 120 prior to use allows the balloon to be inflated to its full diameter and length.

Catheter body 110 is an elongate, flexible member with a proximal end 112 and a distal end 114. One or more ports may be located at proximal end 112 to allow for the insertion of guidewires and to connect, for example, a balloon inflation system to catheter body 110. At distal end 114, catheter body 110 may have a plugging mechanism to prevent leakage into or out of catheter body 110, while allowing clear passage for a guidewire through a guidewire lumen 128. Distal end 114 of catheter body 110 is inserted first into the vasculature of the body through, for example, the femoral artery in the leg where it can be guided into more delicate vasculature including arteries within the human heart. Treatment of vascular conditions may include the prevention or correction of various ailments and deficiencies associated with the cardiovascular system, the cerebrovascular system, urogenital systems, biliary conduits, abdominal passageways and other biological vessels within the body. Catheter body 110 has a catheter sidewall 116, and an outer surface 118 onto which inflatable balloon 120 is attached.

Inflatable balloon 120, which is a flexible and expandable thin-walled tubular member, attaches near distal end 114 of catheter body 110. Inflatable balloon 120 comprises an elastic material such as polyurethane, polyethylene terephthalate (PET), or a thermoplastic elastomer, as is currently known in the art. Inflatable balloon 120 has a length, for example, of 8 millimeters to over 40 millimeters.

The proximal and distal ends of inflatable balloon 120 are attached to catheter body 110 using suitable adhesives, epoxies, glues, heat bonding, collars, bands, or other suitable attachment mechanisms. An interior region 124 between an outer surface 118 of catheter body 110 and an inner surface of an exterior wall of inflatable balloon 120 may be filled with an inflation fluid such as dilute contrast fluid or saline solution to pressurize and enlarge the exterior wall of the balloon. Inflation fluid injected at proximal end 112 of catheter body 110 travels through an inflation lumen 126 inside catheter body 110, through an inflation hole 122 formed in catheter sidewall 116 between inflatable balloon 120 and inflation lumen 126, and into interior region 124 of inflatable balloon 120 to inflate the balloon. To deflate the balloon, inflation fluid is removed from interior region 124 of inflatable balloon 120 through inflation hole 122 and inflation lumen 126 using, for example, a pump within the balloon inflation system or through elastic restoring forces generated by inflatable balloon 120 and any restrictors 130 surrounding the balloon.

Prior to inflation or when deflated, inflatable balloon 120 may be appreciably compressed against outer surface 118 of catheter body 110. When compressed, inflatable balloon 120 has a small diameter that allows insertion into and extraction from the sometimes small, tortuous vasculature of the body. Restrictors 130, elastic sleeves, or an unexpanded stent may be used to maintain inflatable balloon 120 in a compressed state until inflated.

Inflatable balloon 120 expands and inflates in response to increasing pressure from the inflation fluid. As inflatable balloon 120 expands, inflatable balloon 120 presses, for example, against the walls of a restricted vessel to reduce constrictions within the blood vessel, or against a stent framework to expand and deploy a stent within the vessel. As inflation fluid is injected through inflation hole 122, inflatable balloon 120 enlarges or unfolds to a nominally tubular shape, and once inflatable balloon 120 reaches its tubular shape, additional pressure applied to interior region 124 has minimal effect on further enlargements of the balloon diameter. However, with one or more restrictors 130 in place, the size of inflatable balloon 120 is controlled in diameter and length.

Restrictors 130 are tubular members placed around the circumference of inflatable balloon 120. A set of restrictors 130 may include a range of restrictor diameters that provide and limit inflatable balloon 120 to different diameters of radial expansion and to different expandable lengths based on the position of the restrictors and the restrictor diameters. Multiple restrictors 130 may be placed one on top of another, with the smallest restrictor diameter defining the allowable balloon diameter. One or more restrictors 130 may be moved or pulled off inflatable balloon 120 to vary and control the diameter and length of the balloon during inflation. Restrictors 130 may be frictionally coupled to inflatable balloon 120 to retain them on the balloon during storage and use, yet designed to allow freedom to be moved or removed as desired.

Restrictors 130 are formed from one or more layers of expandable material such as polyurethane, silicone, copolymers of polyurethane and silicone, natural rubber, synthetic rubber, a thermoplastic polyamide, nylon, latex, polyethylene, polyisoprene, polyisobutylene, polyethylene terephthalate (PET), polyethylene, polytetrafluoroethylene (PTFE), expanded polytetrafluoroethylene (ePTFE), an elastane, a thermoplastic elastomer, a woven polymeric fabric, an expandable polymeric sheet, an elastomeric material, a semi-compliant polymer, or combinations thereof. The wall thickness of restrictors 130 is thin to maintain a low profile when placed on inflatable balloon 120, though sufficient to maintain the desired restrictor diameter with large inflation pressure. A composite restrictor 130 may be formed from a first material with high elasticity that keeps the restrictor profile small and a second material that provides the desired hoop strength when the balloon is expanded.

Restrictors 130 are readily expanded to a restrictor diameter when a balloon disposed thereunder is inflated. Once restrictors 130 expand to the restrictor diameter, further increases in balloon pressure result in minimal increases in the outside diameter of restrictor 130, thus limiting the expandable diameter of inflatable balloon 120 to the restrictor diameter. In some cases, one or more restrictors 130 have an elastic characteristic that tends to compress inflatable balloon 120 onto the catheter while deflating or when stored. Restrictors 130 may be attached to outside surface 118 of catheter body 110 at one or more points to retain their position until moved or removed.

Restrictor removal elements 132 such as tabs, flaps, threads, strings, threads, or wires may be formed with or attached to restrictors 130 to aid in moving and removing one or more restrictors 130 from inflatable balloon 120. Restrictor removal elements 132 may be color-coded to aid in identification when selecting and moving restrictors 130. Restrictor removal elements 132 may be formed as a unitary member with restrictor 130. A lubricant may be positioned between each of restrictors 130 and between the innermost restrictor 130 and the exterior surface of inflatable balloon 120 to aid in sliding restrictors 130 during use.

While a bitumen catheter body 110 with side-by-side inflation lumen 126 and guidewire lumen 128 is illustrated in FIG. 1, the present invention is applicable to a coaxial catheter body 110 having a flexible inner tube that serves as a guidewire lumen and a flexible outer tube coaxially configured with the flexible inner tube. The inner tube and the outer tube cooperate to form an annular inflation lumen between an outside surface of the interior tube and an interior surface of the outer tube to allow flow of inflation fluid through the annular inflation lumen into and out from inflatable balloon 120.

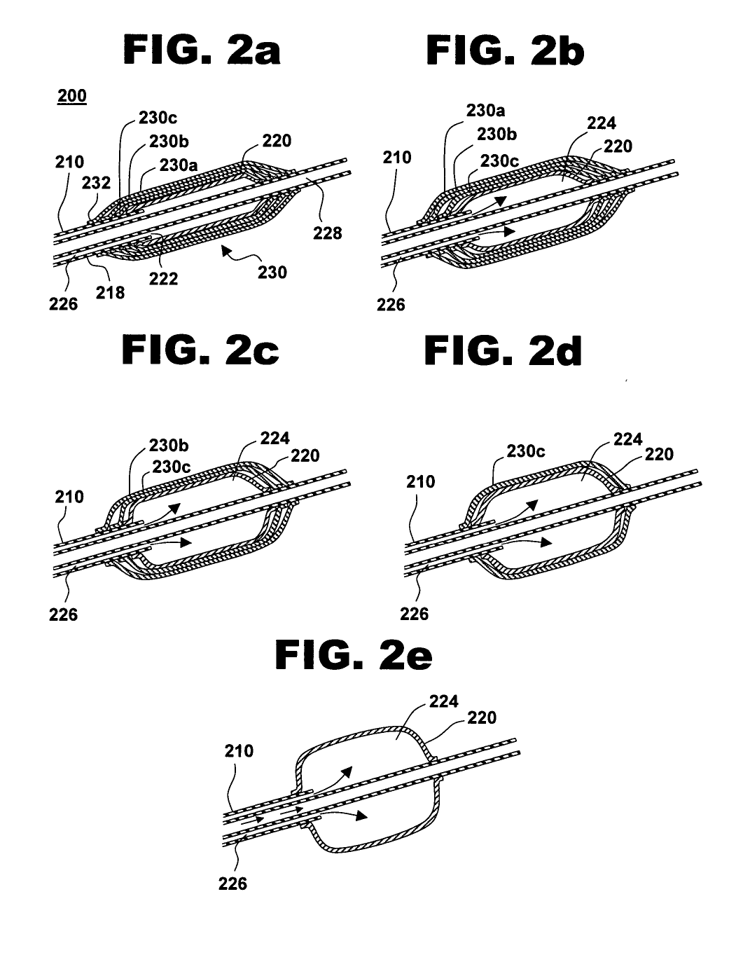

FIG. 2a shows a longitudinal cross-sectional view of a portion of a balloon catheter with a plurality of concentrically disposed restrictors, in accordance with one embodiment of the present invention at 200. Restrictors 230a, 230b and 230c are coupled at each end to an outer surface 218 of a coaxial catheter body 210, covering an inflatable balloon 220. The proximal ends of restrictors 230 are attached to an outer tube of coaxial catheter body 210, and the distal ends of restrictors 230 are attached to an inner tube of coaxial catheter body 210. A guidewire lumen 228 is formed by the inner tube of coaxial catheter body 210. An annular inflation lumen 226 is formed between an interior surface of the outer tube and an exterior surface of the inner tube. An annular inflation hole 222 is formed at the distal end of the outer tube adjacent to inflatable balloon 220, allowing inflation fluid to flow into and out from inflatable balloon 220. Restrictors 230 may be secured tightly or loosely to outer surface 218 of catheter body 210. For example, an epoxy or other adhesive is used to adhere the proximal ends, distal ends, or both ends of restrictors 230 to catheter body 210. In another example, the elasticity of the restrictor material provides sufficient force to be maintained at the desired position. In another example, an elastic sleeve (not shown) may be placed over the set of restrictors 230 to compress restrictors 230 and inflatable balloon 220 against catheter body 210.

Inflation fluid injected through an annular inflation lumen 226 within catheter body 210 traverses annular inflation hole 222 into an interior region 224 of inflatable balloon 220 to inflate the balloon. Restrictors 230 have a predefined outer diameter to which the restrictors can be enlarged, referred to herein as the restrictor diameter. Positioned over an inflatable balloon 220 attached to catheter body 210, restrictors 230 restrict the diameter to which inflatable balloon 220 can be expanded. Each restrictor 230a, 230b and 230c have a predetermined restrictor diameter such as 2.0 millimeters (mm), 3.0 mm, and 4.0 mm, respectively. When inflatable balloon 220 is deflated or otherwise collapsed, the outer profile of the restrictors and underlying balloon is smaller than the smallest restrictor diameter, limited by the outer diameter of catheter body 210 and the thickness of the restrictors and balloon.

More than one restrictor can be in place over inflatable balloon 220. In one example, three restrictors with three different diameters are concentrically disposed on inflatable balloon 220. The outer restrictor 230a has the smallest restrictor diameter of 2.0 mm. The middle restrictor 230b has a larger restrictor diameter of 3.0 mm, and the inner restrictor 230c has the largest restrictor diameter of 4.0 mm. Inflatable balloon 220 has, in this example, a balloon diameter when expanded of 5.0 mm. With restrictors 230a, 230b and 230c in place, inflatable balloon 220 cannot be expanded beyond the restrictor diameter of 2.0 mm, limited by the restrictor diameter of restrictor 230a as inflatable balloon 220 enlarges.

One or more restrictors can be axially translated or removed prior to inflation of inflatable balloon 220 to control the expansion capability of inflatable balloon 220. For example, outer restrictor 230a may be axially displaced towards the proximal end or the distal end of catheter body 210, or physically removed from catheter body 210 such as by pulling, pushing, sliding or tearing. When inflatable balloon 220 is inflated with outer restrictor 230a removed, the balloon diameter is limited by middle restrictor 230b to approximately 3.0 mm in this example. When restrictors 230a and 230b are axially translated or removed from inflatable balloon 220, inflatable balloon 220 may be inflated to a balloon diameter of 4.0 mm. When restrictors 230a, 230b and 230c are axially translated or removed from inflatable balloon 220, inflatable balloon 220 may be inflated to a balloon diameter of 5.0 mm.

In another example, eight restrictors are placed over a 6.0 mm inflatable balloon, with restrictor diameters of 2.0 mm, 2.5 mm, 3.0 mm, 3.5 mm, 4.0 mm, 4.5 mm, 5.0 mm, and 5.5 mm, thereby limiting balloon expansion to 2.0 mm. With the outer restrictor removed, the balloon diameter is limited to 2.5 mm. With the second restrictor removed, the balloon diameter is limited to 3.0 mm. With the third restrictor removed, the balloon diameter is limited to 3.5 mm. With the fourth restrictor removed, the balloon diameter is limited to 4.0 mm. With the fifth restrictor removed, the balloon diameter is limited to 4.5 mm. With the sixth restrictor removed, the balloon diameter is limited to 5.0 mm. With the seventh restrictor removed, the balloon diameter is limited to 5.5 mm. With all restrictors removed or at least axially displaced along catheter body 210 away from inflatable balloon 220, the balloon diameter is limited to 6.0 mm.

To aid in sliding, removing, or detaching restrictors 230, a restrictor removal element 232 such as a tab, a flap, a string, a thread, or a wire is coupled to each restrictor 230. Restrictor removal element 232 and restrictor 230 may be formed as a unitary member. A lubricant or other slide enhancer may be placed between the each of the restrictors and between the restrictors and the outer wall of the balloon. For example, a user's fingers may be used to grab a tab or a flap to slide or detach a selected restrictor. The tab or flap may be formed with the restrictor removal element as a unitary member, such that when the tab or flap is pulled, the restrictor slides readily. In another example, a string or thread coupled to each restrictor 230 may be pulled to slide or remove a selected restrictor. In cases where restrictors 230 are slid or removed, restrictors 230 may be attached to at least one point to catheter body 210 so that sliding or removing one of the restrictors does not appreciably move the others. For example, a tearing mode can be enhanced by forming partial slits in the restrictors close to the attachment points. Partial slits or perforations may be used to retain restrictors 230 in place until intentionally and forcibly moved.

The balloon catheter, although illustrated with a coaxial catheter body design, may have a bi-lumen design having inflation lumen 226 and guidewire lumen 228 side-by-side in an extruded bi-lumen tube or with parallel, longitudinally connected tubes.

FIG. 2b, FIG. 2c, FIG. 2d, and FIG. 2e illustrate longitudinal cross-sectional views of a portion of a balloon catheter with a varying number of concentrically disposed restrictors 230 as inflatable balloon 220 is inflated, in accordance with one embodiment of the present invention. With restrictors 230a, 230b and 230c circumferentially disposed on inflatable balloon 220, inflation fluid flowing through annular inflation lumen 226 of catheter body 210 into interior region 224 inflates inflatable balloon 220 to a diameter limited by the restrictor diameter of restrictor 230a such as 2.0 mm, as shown in FIG. 2b. With restrictor 230a slidably removed from and restrictors 230b and 230c circumferentially disposed on inflatable balloon 220, inflation fluid flowing through annular inflation lumen 226 into interior region 224 inflates inflatable balloon 220 to a balloon diameter limited by the restrictor diameter of restrictor 230b such as 3.0 mm, as shown in FIG. 2c. With restrictors 230a and 230b slidably removed from and restrictor 230c circumferentially disposed on inflatable balloon 220, inflation fluid flowing through annular inflation lumen 226 into interior region 224 inflates inflatable balloon 220 to a balloon diameter limited by the restrictor diameter of restrictor 230c such as 4.0 mm, as shown in FIG. 2d. With restrictors 230a, 230b and 230c slidably removed from inflatable balloon 220, inflation fluid flowing through annular inflation lumen 226 into interior region 224 inflates inflatable balloon 220 to a balloon diameter limited by the diameter of unrestricted inflatable balloon 220 such as 5.0 mm, as shown in FIG. 2e.

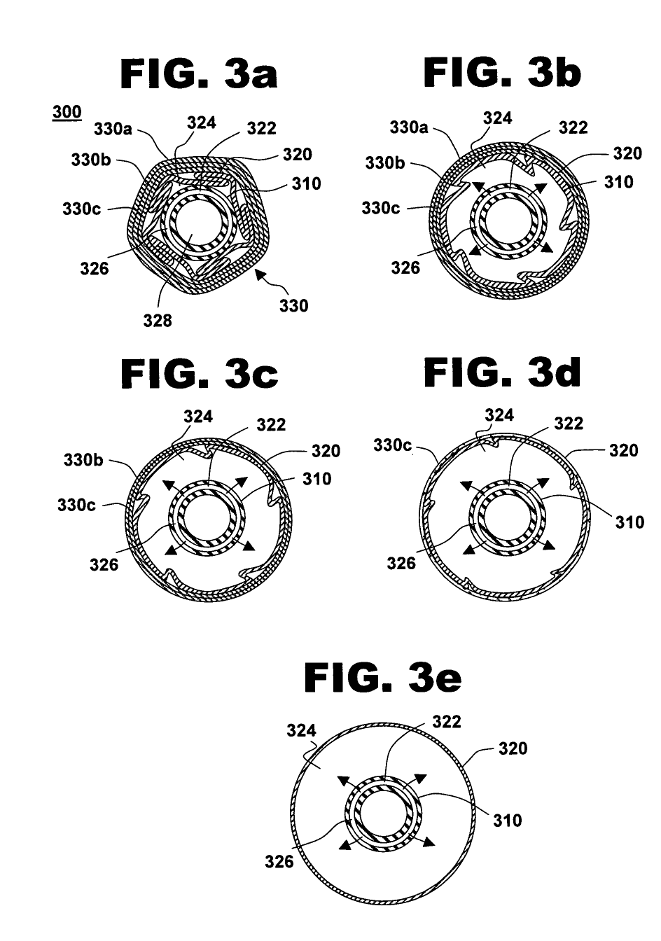

FIG. 3a shows a diametrical cross-sectional of a portion of a balloon catheter with a plurality of concentrically disposed restrictors 330, in accordance with one embodiment of the present invention at 300. Catheter body 310 has an annular inflation lumen 326 and a guidewire lumen 328. An annular inflation hole 322 in a catheter sidewall fluidly connects annular inflation lumen 326 to an interior region 324 between a wall of inflatable balloon 320 and catheter body 310. In this embodiment, three restrictors 330a, 330b and 330c are circumferentially disposed on inflatable balloon 320. As inflation fluid is injected into interior region 324 of inflatable balloon 320, inflatable balloon 320 expands accordingly until restricted by one or more restrictors 330 or by the balloon diameter of inflatable balloon 320. Inflatable balloon 320, which may be folded with one or more pleats, is compressed into an uninflated or deflated state having a small cross section, thereby aiding the insertion of inflatable balloon 320 into and retraction from the body.

FIG. 3b, FIG. 3c, FIG. 3d, and FIG. 3e show diametrical cross-sectional views of a portion of a balloon catheter with an inflated balloon and a plurality of concentrically disposed restrictors 330, in accordance with one embodiment of the present invention. To inflate inflatable balloon 320, fluid is injected through annular inflation lumen 326 and annular inflation hole 322 of catheter body 310 into interior region 324 between a wall of inflatable balloon 320 and catheter body 310.

As inflation fluid is injected into inflatable balloon 320, a set of restrictors 330a, 330b and 330c around inflatable balloon 320 limit the expansion of inflatable balloon 320, the expansion of the balloon diameter being limited by the restrictor diameter of restrictor 330a such as 2.0 mm, as shown in FIG. 3b.

When restrictor 330a is axially displaced or otherwise removed from around inflatable balloon 320, and restrictors 330b and 330c are circumferentially disposed on inflatable balloon 320, inflation fluid expands inflatable balloon 320 to a diameter limited by the restrictor diameter of restrictor 330b such as 3.0 mm, as shown in FIG. 3c.

When restrictors 330a and 330b are axially displaced or otherwise removed from around inflatable balloon 320, and restrictor 330c is circumferentially disposed on inflatable balloon 320, inflatable balloon 320 inflates to a diameter limited by the restrictor diameter of restrictor 330c such as 4.0 mm, as shown in FIG. 3d.

With restrictors 330a, 330b and 330c axially displaced or otherwise removed from around inflatable balloon 320, inflation fluid expands inflatable balloon 320 to a diameter limited by the balloon diameter such as 5.0 mm, as shown in FIG. 3e.

While FIG. 3b, FIG. 3c, FIG. 3d, and FIG. 3e illustrate restrictors of specific sizes, others embodiments use restrictors 330 that limit the balloon to different increasing and decreasing sizes. A number of selectable diameters can be accommodated by the choice of a restrictor diameter and number of restrictors.

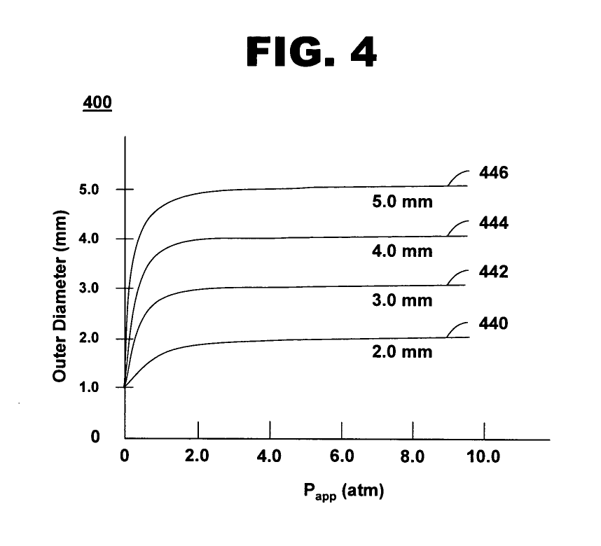

FIG. 4 is a graph of balloon diameter with applied pressure for a variable expansion balloon catheter, in accordance with one embodiment of the present invention at 400. Graph 400 shows the outer diameter of an inflatable balloon with varying combinations of restrictors in place around the inflatable balloon. As the balloon is inflated, the diameter of the balloon quickly changes from the uninflated or deflated state to the expanded state as the balloon unfolds or stretches, limited by the restrictor diameter of one of the restrictors or by the balloon diameter when all the restrictors are removed.

For example, response curve 440 shows the balloon diameter increasing from approximately 1.0 mm in an uninflated or deflated state to a balloon diameter of 2.0 mm, limited by the presence of restrictor with a 2.0 mm restrictor diameter around the inflatable balloon. As pressure is applied to the interior region of the balloon, response curve 442 shows the balloon diameter increasing from approximately 1.0 mm to a diameter of 3.0 mm, limited by the presence of a restrictor with a 3.0 mm restrictor diameter around the inflatable balloon. Response curve 444 shows the balloon diameter limited to a diameter of 4.0 mm, limited by the presence of a restrictor with a 4.0 mm restrictor diameter around the inflatable balloon. Response curve 446 shows a fully expanded balloon, limited by the balloon diameter of 5.0 mm. For fully expanded, non-compliant balloon and restrictor materials, slight increases in balloon diameter may occur with further increases in applied pressure, whereas with compliant and expandable balloon and restrictor materials, further increases in applied pressure will result in larger diameters. The outer diameters obtained at a given applied pressure may be reduced by the rigidity of the vessel walls or by the inclusion of a stent around the outside of the inflation balloon and the restrictors.

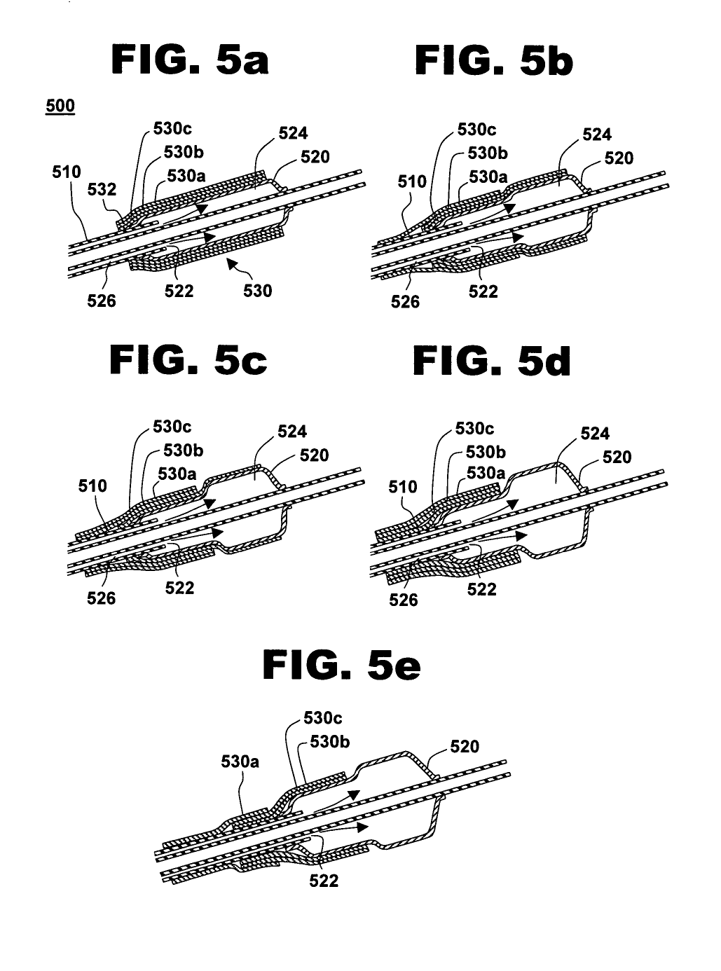

FIG. 5a, FIG. 5b, FIG. 5c, FIG. 5d, and FIG. 5e show longitudinal cross-sectional views of a portion of a balloon catheter with a plurality of concentrically disposed restrictors, in accordance with one embodiment of the present invention at 500. To inflate inflatable balloon 520, fluid is injected through annular inflation lumen 526 and annular inflation hole 522 of catheter body 510 into interior region 524 between catheter body 510 and a wall of inflatable balloon 520. The expandable length of an inflatable balloon 520 that is mounted on a catheter body 510 can be controlled by axially displacing one or more restrictors 530 to limit the expansion of select portions of inflatable balloon 520 to a predetermined balloon diameter when inflation fluid is injected into inflatable balloon 520.

A set of restrictors 530a, 530b and 530c are circumferentially disposed on inflatable balloon 520, and when inflatable balloon 520 is inflated, the balloon diameter is limited to the smallest restrictor diameter such as 2.0 mm, as seen in FIG. 5a. To aid in sliding, removing, or detaching restrictors 530, a restrictor removal element 532 such as a tab, a flap, a string, a thread, or a wire may be coupled to each restrictor 530.

As restrictor 530a is axially displaced towards a proximal end or a distal end of catheter body 510 leaving restrictors 530b and 530c, a portion of inflatable balloon 520 is allowed to expand to a balloon diameter set by restrictor 530b on one side, and limited to a balloon diameter set by restrictor 530a on the other side, as seen in FIG. 5b. For example, an expandable length of inflatable balloon 520 is 10.0 mm with a balloon diameter of 3.0 mm at the distal end, while the remaining portion towards the proximal end is restricted to a balloon diameter of 2.0 mm.

As restrictors 530a and 530b are axially displaced towards a proximal end or a distal end of catheter body 510, a portion of inflatable balloon 520 is allowed to expand to a balloon diameter set by restrictor 530c on one side, and limited to a balloon diameter set by restrictor 530a on the other side, as seen in FIG. 5c. For example, an expandable length at the distal end of inflatable balloon 520 is 10.0 mm with a balloon diameter of 4.0 mm, while the proximal half is restricted to a balloon diameter of 2.0 mm.

As restrictors 530a, 530b and 530c are axially displaced towards a proximal end or a distal end of catheter body 510, a portion of inflatable balloon 520 is allowed to expand to a balloon diameter set by the balloon diameter on one side, and by restrictor 530a on the other side, as seen in FIG. 5d. For example, an expandable length of inflatable balloon 520 is 10.0 mm with a balloon diameter of 5.0 mm, while the other half is restricted to a balloon diameter of 2.0 mm.

To allow more flexibility in selecting the expandable length and expandable diameter of the balloon, restrictors 530a, 530b, and 530c can be axially displaced to control the balloon diameter and expandable length of each side, as seen in FIG. 5e. For example, an expandable length of inflatable balloon 520 is 10.0 mm with a balloon diameter of 5.0 mm on one side, while the other side has a balloon diameter of 3.0 mm.

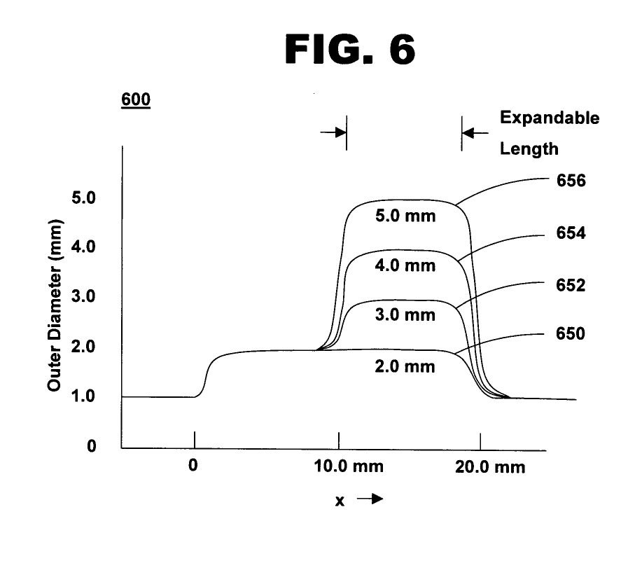

FIG. 6 shows a graph of balloon diameter versus position along the inflated balloon for a variable expansion balloon catheter, in accordance with one embodiment of the present invention at 600. The balloon diameters and lengths correspond with the descriptions of the examples given for FIG. 5a to FIG. 5d. Response curve 650 has three restrictors in place, with the outer restrictor having a restrictor diameter of 2.0 mm resulting in a balloon diameter of 2.0 mm and an expandable length of 20 mm when the inflatable balloon is inflated.

As restrictors are selected and axially displaced with respect to the inflatable balloon, the balloon diameter and balloon length effectively change accordingly. For example, response curve 652 shows an expanded balloon with an expandable length of 10.0 mm and a balloon diameter of 3.0 mm on one side, with a balloon diameter of 2.0 mm on the other side. Response curve 654 shows an expanded balloon with an expandable length of 10.0 mm and a balloon diameter of 4.0 mm on the distal side, with a balloon diameter of 2.0 mm on the proximal side. Response curve 656 shows an expanded balloon with an expandable length of 10.0 mm and a balloon diameter of 5.0 mm on the distal half, with a balloon diameter of 2.0 mm on the proximal half.

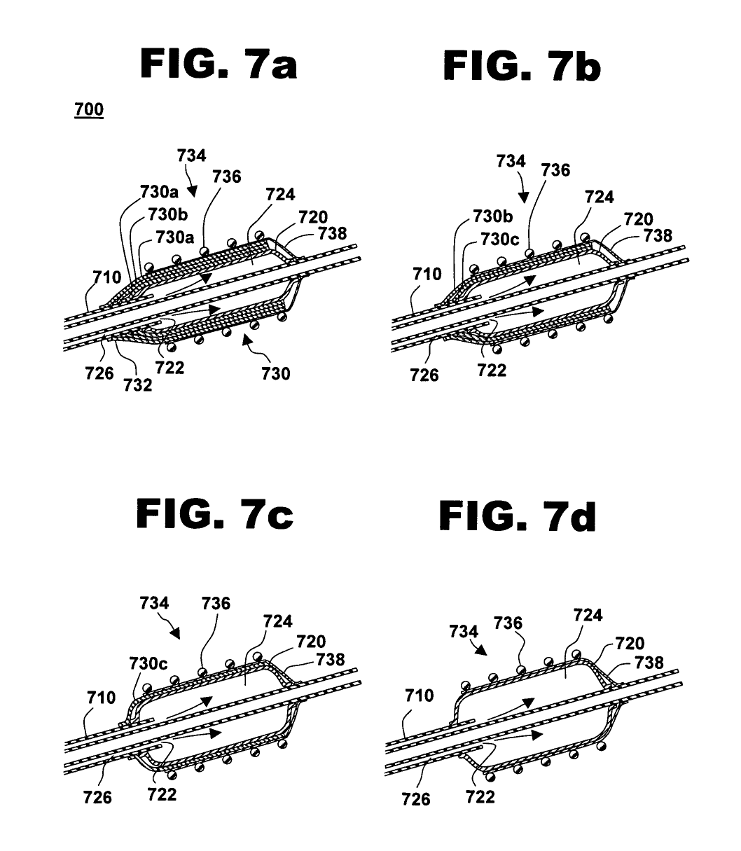

FIG. 7a, FIG. 7b, FIG. 7c and FIG. 7d show longitudinal cross-sectional views of a portion of a variable expansion balloon catheter with a stent for controlled-diameter stent delivery, in accordance with one embodiment of the present invention at 700. A stent 734 including a stent framework 736 is positioned on and coupled to an inflatable balloon 720, with one or more concentrically disposed restrictors 730a, 730b, and 730c positioned between stent framework 736 and inflatable balloon 720. Inflatable balloon 720 inflates when inflation fluid injected through an annular inflation lumen 726 in a catheter body 710 and through an annular inflation hole 722 into an interior region 724 between an outer surface of catheter body 710 and a wall of inflatable balloon 720. As inflation fluid is injected into interior region 724, inflatable balloon 720 expands accordingly, limited by the inclusion of restrictors 730. Restrictors 730 are removable to allow expansion of stent framework 736 to a predetermined stent diameter. A restrictor removal element 732 such as a tab, a flap, a string, a thread, or a wire may be coupled to each restrictor 730 to aid in the selective removal of restrictors 730.

In one embodiment, a retention sheath 738 is positioned between restrictors 730 and stent framework 736 to retain stent 734 on inflatable balloon 720 while various restrictors 730 are being removed. Retention sheath 738 is attached, for example, to a point on catheter body 710 distal to inflatable balloon 720, so that when restrictors 730 are pulled towards a proximal end of catheter body 710, stent 734 remains unmoved.

Restrictors 730 may also be attached at a point on catheter body 710 distal to inflatable balloon 720, so that when one or more selected restrictors 730 are pulled towards a proximal end of catheter body 710, the selected restrictors detach or tear away from catheter body 710 and are removed, while the other restrictors stay in place. In this manner, a restrictor with the smallest diameter may be positioned inside of other restrictors rather than outside, being able to be pulled out without damaging or moving the other restrictors, the balloon, or the stent.

In a first example, stent 734 is enlarged to a stent diameter limited by the restrictor diameter of restrictor 730a such as 2.0 mm, as seen in FIG. 7a. In a second example, stent 734 is enlarged to a balloon diameter limited by the restrictor diameter of restrictor 730b such as 3.0 mm, as seen in FIG. 7b. In a third example, stent 734 is enlarged to a stent diameter limited by the restrictor diameter of restrictor 730c such as 4.0 mm, as seen in FIG. 7c. In a fourth example, stent 734 is enlarged to a stent diameter limited by the balloon diameter such as 5.0 mm, as seen in FIG. 7d. Other stent diameters and the number of selectable diameters can be accommodated by choice of restrictor diameter and number of restrictors between the stent and the balloon.

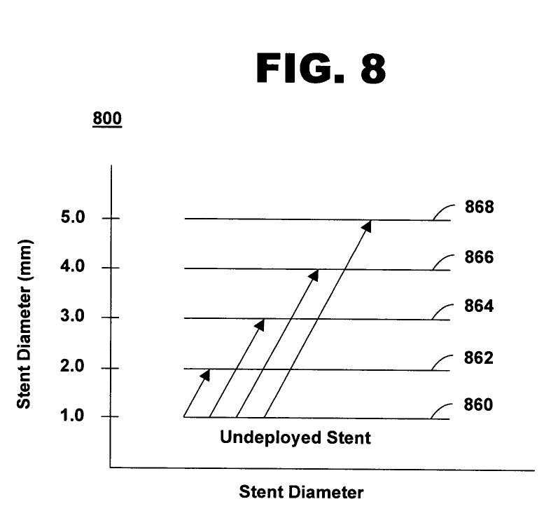

FIG. 8 shows a graphical illustration of stent diameter for a stent deployed with a variable expansion balloon catheter, in accordance with one embodiment of the present invention at 800. Graph 800 shows the stent diameter of a stent deployed with various restrictors in place or removed, corresponding to the examples put forth in FIG. 7a to FIG. 7d.

An exemplary stent diameter 860 corresponds to an undeployed stent with a stent diameter such as 1.0 mm, as placed on the inflatable balloon of the variable expansion balloon catheter. Stent diameter 862 corresponds to a deployed stent with a stent diameter such as 2.0 mm, limited by a restrictor with a restrictor diameter of 2.0 mm. Stent diameter 864 corresponds to a deployed stent with a stent diameter of 3.0 mm, limited by a restrictor with a restrictor diameter of 3.0 mm. Stent diameter 866 corresponds to a deployed stent with a stent diameter of 4.0 mm, corresponding to a restrictor diameter of 4.0 mm. Stent diameter 868 corresponds to a deployed stent with a stent diameter of 5.0 mm, corresponding to a balloon diameter of 5.0 mm. Other deployed stent diameters may be predetermined by pre-selection of restrictors. Stents may be positioned on the selected restrictors or set of restrictors when initially assembled or during clinical use.

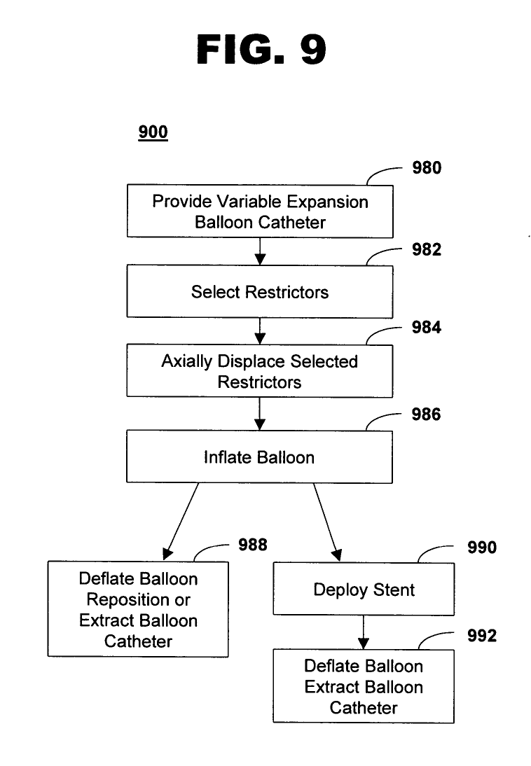

FIG. 9 is a flow chart of a method for treating a vascular condition, in accordance with one embodiment of the present invention at 900. Vascular treatment method 900 shows various steps for using a variable expansion balloon catheter.

A variable expansion balloon catheter is provided, as seen at block 980. The variable expansion balloon catheter includes a plurality of concentrically disposed restrictors circumferentially disposed on an inflatable balloon of a balloon catheter. Axially translatable restrictors allow the inflatable balloon to expand up to a predetermined size.

At least one of the restrictors is selected, based on a predetermined balloon size, as seen at block 982. The predetermined balloon size may include the balloon diameter, the expandable balloon length, or a combination thereof.

The selected restrictors are axially displaced with respect to the inflatable balloon, as seen at block 984. In one example, one or more restrictors are slidably moved along the balloon catheter to reveal a portion of the inflatable balloon and control the expandable length of the balloon. In this multiple-diameter configuration, either the proximal end or the distal end of the inflatable balloon may be selected to have the larger diameter depending on the procedure. The transition between the larger and smaller diameters may be made at any point along the working length of the inflatable balloon. In another example, one or more restrictors are slid away from the inflatable balloon towards a proximal end of the catheter, such that the selected restrictors remain on the catheter yet are separated by a distance from the inflatable balloon. The balloon is inflatable to a pre-selected balloon diameter based on the restrictor diameters of the restrictors remaining on the inflatable balloon. In another example, the selected restrictors are completely removed from the variable expansion balloon catheter.

The selected restrictors may be manually pulled by pulling a restrictor removal element such as a tab, a flap, a string, a thread, or a wire attached to the selected restrictors. In another example, a medical practitioner would select one, two or more restrictors, slide the selected restrictors over an end of the catheter body, and position the restrictors accordingly over an inflatable balloon prior to balloon inflation to achieve the desired balloon diameter and expandable length.

The inflatable balloon is inflated to the predetermined balloon size based on the axial displacement of the selected restrictors, as seen at block 986. In one example, the inflatable balloon is allowed to expand to a predetermined balloon diameter when the inflatable balloon is inflated. In another example, the expandable length of the inflatable balloon is controlled when inflating the inflatable balloon.

The balloon is deflated and the variable expansion balloon catheter is repositioned within or extracted from the body, as seen at block 988. In an exemplary medical treatment using balloon angioplasty, an inflatable balloon is positioned in a vessel, expanded to the predetermined balloon diameter to enlarge the vessel, and then deflated. The variable expansion balloon catheter may be repositioned and the inflatable balloon can be expanded several times as desired at locations within one or more blood vessels, and then extracted from the body.

Some medical cases may require that an additional restrictor be selected to increase the expandable diameter or to adjust the expandable length of the inflatable balloon. One or more restrictors may be axially displaced or removed from the inflation balloon while the balloon catheter is outside the body, after which the balloon catheter may be re-inserted into the body. The practitioner can reposition the balloon at other treatment areas needing angioplasty, and inflate the catheter balloon to the larger balloon diameter. After withdrawal, yet another restrictor can be displaced or removed to allow the inflatable balloon to be expanded to an even larger diameter. The balloon catheter may be re-inserted into the body to continue the angioplastic maneuvers. When desired, the expandable length of the inflatable balloon can be adjusted, for example, by repositioning or partially removing selected restrictors from the inflatable balloon. In other medical cases such as dilating a bifurcate lesion, two variable expansion balloon catheters with pre-selected balloon lengths and diameters may be concurrently employed.

Alternatively, the variable expansion balloon catheter may be used to deploy a stent including a stent framework, as seen at block 990. One or more restrictors are selected based on a predetermined balloon size needed to expand a stent that is coupled to the inflatable balloon. The stent is expanded to a predetermined stent diameter based on the axial displacement or removal of the selected restrictors. After the stent is deployed, the balloon is deflated and the balloon catheter is extracted, as seen at block 992.

Variations and alterations in the design, manufacture and use of the restrictors are apparent to one skilled in the art, and may be made without departing from the spirit and scope of the present invention. While the embodiments of the invention disclosed herein are presently preferred, various changes and modifications can be made without departing from the spirit and scope of the invention. The scope of the invention is indicated in the appended claims, and all changes that come within the meaning and range of equivalents are intended to be embraced therein.

1. A system for treating a vascular condition, comprising:
a catheter body;
an inflatable balloon disposed on the catheter body;
a plurality of concentrically disposed restrictors circumferentially disposed on the inflatable balloon, wherein one or more restrictors are at least partially removable to allow expansion of the inflatable balloon to a predetermined size; and
inflation means to inflate the inflatable balloon, the inflation means coupled to a proximal end of the catheter body.

2. The system of claim 1 wherein the catheter body includes an inflation lumen and an inflation hole adjacent to the inflatable balloon, wherein the inflation lumen and the inflation hole allow inflation fluid from the inflation means to inflate the inflatable balloon.

3. The system of claim 1 wherein the one or more restrictors are partially removable to allow expansion of the inflatable balloon to a predetermined balloon diameter.

4. The system of claim 1 wherein the one or more restrictors are partially removable to control an expandable length of the inflatable balloon.

5. The system of claim 1 wherein the one or more restrictors are frictionally coupled to the inflatable balloon.

6. The system of claim 1 wherein each restrictor is attached to the catheter body at at least one point on an outside surface of the catheter body.

7. The system of claim 1 further comprising:
a restrictor removal element attached to each restrictor.

8. The system of claim 7 wherein the restrictor removal element and the restrictor are formed as a unitary member.

9. The system of claim 7 wherein the restrictor removal element is selected from the group consisting of a tab, a flap, a string, a thread, and a wire.

10. The system of claim 1 wherein the one or more restrictors comprise a material selected from the group consisting of polyurethane, silicone, a copolymer of polyurethane and silicone, natural rubber, synthetic rubber, a thermoplastic polyamide, nylon, latex, polyethylene, polyisoprene, polyisobutylene, polyethylene terephthalate, polyethylene, polytetrafluoroethylene, expanded polytetrafluoroethylene, an elastane, a thermoplastic elastomer, a woven polymeric fabric, an expandable polymeric sheet, an elastomeric material, a semi-compliant polymer, and a combination thereof.

11. The system of claim 1 further comprising:
a stent including a stent framework operably coupled to the inflatable balloon, wherein the one or more restrictors are removable to allow expansion of the stent framework to a predetermined stent diameter.

12. A method of treating a vascular condition, comprising:
providing a variable expansion balloon catheter including a plurality of concentrically disposed restrictors circumferentially disposed on an inflatable balloon;
selecting at least one of the restrictors based on a predetermined balloon size;
axially displacing the selected restrictors with respect to the inflatable balloon; and
inflating the inflatable balloon to the predetermined balloon size based on the axial displacement of the selected restrictors.

13. The method of claim 12 wherein axially displacing the selected restrictors comprises sliding the selected restrictors towards a proximal end of the variable expansion balloon catheter.

14. The method of claim 12 wherein axially displacing the selected restrictors comprises removing the selected restrictors from the variable expansion balloon catheter.

15. The method of claim 12 wherein axially displacing the selected restrictors comprises manually pulling at least one restrictor removal element attached to the selected restrictors.

16. The method of claim 12 wherein inflating the inflatable balloon to the predetermined balloon size comprises allowing expansion of the inflatable balloon to a predetermined balloon diameter when the inflatable balloon is inflated.

17. The method of claim 12 wherein inflating the inflatable balloon to the predetermined balloon size comprises controlling an expandable length of the inflatable balloon.

18. The method of claim 12 further comprising:
expanding a stent including a stent framework, the stent framework operably coupled to the inflatable balloon; wherein the stent is expanded to a predetermined stent diameter based on the axial displacement of the selected restrictors.

19. A balloon catheter, comprising:
a catheter body;
an inflatable balloon disposed on the catheter body; and
a plurality of concentrically disposed restrictors circumferentially disposed on the inflatable balloon, wherein one or more restrictors are axially translatable to allow expansion of the inflatable balloon to a predetermined size.

20. The balloon catheter of claim 19 wherein the catheter body includes an inflation lumen and an inflation hole adjacent to the inflatable balloon, wherein the inflation lumen and the inflation hole allow inflation of the inflatable balloon when an inflation fluid is injected into the inflation lumen at a proximal end of the balloon catheter.

21. The balloon catheter of claim 19 wherein the one or more restrictors are axially translatable to allow expansion of the inflatable balloon to a predetermined balloon diameter.

22. The balloon catheter of claim 19 wherein the one or more restrictors are axially translatable to control an expandable length of the inflatable balloon.

23. The balloon catheter of claim 19 wherein the one or more restrictors are frictionally coupled to the inflatable balloon.

24. The balloon catheter of claim 19 wherein each restrictor is attached to the catheter body at least one point on an outside surface of the catheter body.

25. The balloon catheter of claim 19 wherein the one or more restrictors comprise a material selected from the group consisting of polyurethane, silicone, a copolymer of polyurethane and silicone, natural rubber, synthetic rubber, a thermoplastic polyamide, nylon, latex, polyethylene, polyisoprene, polyisobutylene, polyethylene terephthalate, polyethylene, polytetrafluoroethylene, expanded polytetrafluoroethylene, an elastane, a thermoplastic elastomer, a woven polymeric fabric, an expandable polymeric sheet, an elastomeric material, a semi-compliant polymer, and a combination thereof.

26. The balloon catheter of claim 19 further comprising:
a restrictor removal element attached to each restrictor.

27. The balloon catheter of claim 26 wherein the restrictor removal element and the restrictor are formed as a unitary member.

28. The balloon catheter of claim 26 wherein the restrictor removal element is selected from the group consisting of a tab, a flap, a string, a thread, and a wire.

29. The balloon catheter of claim 19 further comprising:
a stent including a stent framework operably coupled to the inflatable balloon, wherein the one or more restrictors are removable to allow expansion of the stent framework to a predetermined stent diameter.
